(19) United States
(12) Patent Application Publication
(10) Pub. No.: US 2022/0010180 A1
(43) Pub. Date: Jan. 13, 2022

(54) ELECTRICALLY CONDUCTIVE ADHESIVE

(71) Applicant: Heraeus Deutschland GmbH & Co. KG, Hanau (DE)

(72) Inventors: Stefan SCHIBLI, Hanau (DE); Christian NEUMANN, Hanau (DE); Sebastian VOGT, Wehrheim (DE); Andreas REISINGER, Hanau (DE)

(73) Assignee: Heraeus Deutschland GmbH & Co. KG, Hanau (DE)

(21) Appl. No.: 17/372,975

(22) Filed: Jul. 12, 2021

(30) Foreign Application Priority Data
Jul. 13, 2020 (EP) 20 185 439.5

(51) Int. Cl. C09J 133/06; C09J 9/02; C09J 4/06; C09J 5/00; A61N 1/375

(52) U.S. Cl. CPC C09J 133/062; C09J 9/02; C09J 2433/00; C09J 5/00; A61N 1/3754; C09J 4/06

(57) ABSTRACT

One aspect refers to an electrically conductive adhesive including a) a (meth)acrylate monomer, b) a polymer being soluble in the (meth)acrylate monomer, c) a biocompatible metal having a median particle size d₅₀ of below 50 μm, and d) a polymerization initiator. One aspect also refers to a kit for preparing an electrically conductive adhesive, to an implantable medical device including such an electrically conductive adhesive, or a cured form thereof, and to the use of such an electrically conductive adhesive.

### CROSS-REFERENCE TO RELATED APPLICATION

This Utility Patent Application claims priority to European Application No. 20 185 439.5 filed on Jul. 13, 2020, which is incorporated herein by reference.

### TECHNICAL FIELD

One aspect relates to an electrically conductive adhesive, a kit for an electrically conductive adhesive, an implantable device including the electrically conductive adhesive or a cured form thereof, and the use of an electrically conductive adhesive for connecting electronic parts in an implantable medical device.

### BACKGROUND

Active implantable medical devices contain electrical contact elements which connect the electronics of the implant to wires, electrodes or electrode arrays. The wires or electrodes are in contact with nerves or tissues of an organ or a specific body part of the patient carrying the implant. An electrical impulse or electrical current can be passed from the electronics of the implant to the nerves or tissues of the patient for microstimulating this specific part of the body.

The connections between the electronics of the implant and the wires or electrodes have to be durable and reliable in function as any failure may lead to severe consequences for the health of the patient. Furthermore, the materials used for connecting the parts of the implant have to be safe from a medical perspective for a long-term use in the body of the patient.

The connection of the electronics of the implant with wires or electrodes is usually further complicated by the fact that the electronics of the implant have to be secured in a hermetically sealed housing to avoid contact with body fluids. The electronics of the implant are then connected to the wires or electrodes via a feedthrough which electrically connects the interior of the housing to the outside. The electrical connection of wires or electrodes to a feedthrough, especially to a ceramic/metal composite (cermet) feedthrough, is challenging.

In the state of the art, electrical connections in active medical implants are often created by welding or brazing processes. As such processes require the use of high temperature, the choice of the materials of the implant can be limited. Furthermore, use of high temperature for assembling the implantable device can lead to fractures or microfractures in the materials of the device, which may ultimately limit its lifetime. Another possibility to create electrical connections in implantable devices is by mechanically fasten, e.g. crimping, electric pieces to one another. As the trend in active implantable medical devices is towards miniaturization, mechanically connecting electrical components is very difficult, and will become even more so in the near future. Further to this, a mechanical connection has a risk of fatigue and therefore creates a risk of failure, which should be avoided.

In view of the above, there is a continuing need in the art for new electrical connections, which may be used in implantable medical devices, especially active implantable medical devices. In particular, the electrical connections should be biocompatible to allow for a long-term use in the body of a patient, the electrical connection should be suitable for miniaturization, and should be applicable under comparatively mild conditions.

Therefore, the present embodiments are directed to the provision of a new electrical connection, which may be used in implantable medical devices.

### SUMMARY

One embodiment provides an electrically conductive adhesive including

a) a (meth)acrylate monomer,

b) a polymer being soluble in the (meth)acrylate monomer,

c) a biocompatible metal having a median particle size d₅₀ of below 50 μm, and

d) a polymerization initiator.

The inventors found that a composition including a (meth)acrylate monomer, a polymer being soluble in the (meth)acrylate monomer, a biocompatible metal having a median particle size d₅₀ of below 50 μm, and a polymerization initiator, provides an electrically conductive adhesive. The inventive adhesive can be useful for connecting electrical parts e.g. in an implantable medical device.

Furthermore, it has been found that an electrically conductive adhesive can successfully be based on a biocompatible metal and a (meth)acrylate monomer as a polymerizable pre-cursor of the adhesive. (Meth)acrylate monomers, and their polymers or copolymers, are used in the art for preparing bone cements, which may be applied e.g. for fixing endoprostheses in the human skeleton. Therefore, (meth)acrylate monomers, and especially their polymers or copolymers, also illustrate biocompatibility. In view thereof, the inventive adhesive successfully combines the function of a conductive adhesive with a suitability for use in implantable medical device. In another aspect of one embodiment, a kit for preparing an electrically conductive adhesive is provided. The kit includes a component A and a component B,

wherein component A includes a (meth)acrylate monomer, and

wherein component B includes a polymer being soluble in the (meth)acrylate monomer of component A, a biocompatible metal having a median particle size d₅₀ of below 50 μm, and a polymerization initiator.

In yet another aspect of one embodiment, an implantable medical device is provided. The implantable medical device includes at least two electronic parts,

wherein the at least two electronic parts are connected by

a) the electrically conductive adhesive according to any one of claims 1 to 12, or a cured form thereof, or

b) the electrically conductive adhesive prepared from the kit according to claim 13, or a cured form thereof.

A further aspect of one embodiment refers to the use of an electrically conductive adhesive, or a kit according to one embodiment, for connecting at least two electronic parts in an implantable medical device.

It should be understood that for the purposes of the present embodiments, the following terms have the following meanings:

The term “(meth)acrylate” is meant to encompass the options of “methacrylate” and “acrylate”. “Methacrylate” relates to a methacrylic acid ester and “acrylate” to an acrylic acid ester.

The term “biocompatible” in the meaning of the present embodiments is meant to refer to a material which is considered by a person skilled in the art to be safe when being in contact with a living organism (e.g. a human) over a longer period of time (e.g. when used in an implantable medical device).

The “median particle size d₅₀” indicates a diameter value such that 50% of the particles have a diameter of less than this value. A “particle size d₉₀” indicates a diameter value such that 90% of the particles have a diameter of less than this value. In one embodiment, the particle size as defined herein is measured as a “volume-based” particle size distribution. For example, a “volume-based” median particle size d₅₀ indicates a diameter value such that 50% by volume of the particles have a diameter of less than this value. The volume-based particle size distribution can be measured by laser diffraction, e.g. using a Malvern Mastersizer 2000 or 3000 laser diffraction system.

Where an indefinite or definite article is used when referring to a singular noun, e.g., “a”, “an” or “the”, this includes a plural of that noun unless anything else is specifically stated.

Where the term “comprising” is used in the present description and claims, it does not exclude other elements. For the purposes of the present embodiments, the terms “essentially consisting of” and “consisting of” are considered to be a preferred embodiments of the term “comprising”. If hereinafter a group is defined to comprise at least a certain number of embodiments, this is also to be understood to disclose a group, which preferably essentially consists of only of these embodiments, or preferably consists of only of these embodiments.

Terms like “obtainable” or “definable” and “obtained” or “defined” are used interchangeably. This, for example, means that, unless the context clearly dictates otherwise, the term “obtained” does not mean to indicate that, for example, an embodiment must be obtained by, for example, the sequence of steps following the term “obtained” though such a limited understanding is always included by the terms “obtained” or “defined” as a preferred embodiment.

Whenever the terms “including” or “having” are used, these terms are meant to be equivalent to “comprising” as defined hereinabove.

### DETAILED DESCRIPTION

In the following Detailed Description, reference is made to the accompanying drawings, which form a part hereof, and in which is illustraten by way of illustration specific embodiments in which the embodiment may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present embodiment. The following detailed description, therefore, is not to be taken in a limiting sense, and the scope of the present embodiment is defined by the appended claims.

It is to be understood that the features of the various exemplary embodiments described herein may be combined with each other, unless specifically noted otherwise.

### Electrically Conductive Adhesive

One embodiment provides an electrically conductive adhesive including

a) a (meth)acrylate monomer,

b) a polymer being soluble in the (meth)acrylate monomer,

c) a biocompatible metal having a median particle size d₅₀ of below 50 μm, and

d) a polymerization initiator.

The electrically conductive adhesive according to one embodiment includes a (meth)acrylate monomer. The (meth)acrylate monomer is polymerizable. Thus, the (meth)acrylate monomer shall polymerize when the adhesive is cured, e.g. after its application to a substrate, and initiation of the polymerization. The (meth)acrylate monomer is in one embodiment polymerizable in a radical polymerization. After polymerization and/or curing of the adhesive, the (meth)acrylate, together with other organic compounds of the adhesive, forms the matrix of the adhesive into which the biocompatible metal is embedded.

The (meth)acrylate monomer may be a (meth)acrylate monomer with a boiling point above 90° C., in one embodiment in the range of 90 to 200° C., and in one embodiment in the range of 95 to 200° C.

The (meth)acrylate monomer may have a molecular weight of below 1000 g/mol, in one embodiment from 90 to 1000 g/mol, in one embodiment from 90 to 400 g/mol, and in one embodiment from 100 to 200 g/mol.

The (meth)acrylate monomer and/or the polymer derived from the (meth)acrylate monomer is in one embodiment a biocompatible material, and in one embodiment a biocompatible material according to ISO 10993.

The (meth)acrylate monomer may be a methacrylate monomer or an acrylate monomer, and in one embodiment the (meth)acrylate monomer is a methacrylate monomer.

The (meth)acrylate monomer may be selected from the group consisting of alkyl (meth)acrylate monomers, diol di(meth)acrylate monomers, and mixtures thereof. It is preferred that the (meth)acrylate monomer is selected from the group consisting of alkyl (meth)acrylate monomers.

The alkyl group of the alkyl (meth)acrylate monomer may be substituted or unsubstituted, but it is preferred that the alkyl group is an unsubstituted alkyl group. The alkyl group of the (meth)acrylate monomer may be saturated or unsaturated, and in one embodiment is saturated. The alkyl group of the (meth)acrylate monomer may be branched or unbranched, and in one embodiment is unbranched. The diol group of the diol di(meth)acrylate may be substituted or unsubstituted, saturated or unsaturated, and branched or unbranched. In one embodiment, the diol group is unsubstituted, saturated and unbranched.

In one embodiment, the alkyl (meth)acrylate monomer is a C₁-C₂₀-alkyl (meth)acrylate monomer, in one embodiment a C₁-C₁₀-alkyl (meth)acrylate monomer, in one embodiment a C₁-C₆-alkyl (meth)acrylate monomer, and in one embodiment a C₁-C₄-alkyl (meth)acrylate monomer.

The diol di(meth)acrylate monomer is in one embodiment a C₁-C₁₀-diol di(meth)acrylate such as ethyleneglycol dimethacrylate, butane-1,4-diol dimethacrylate or hexane-1,6-diol dimethacrylate.

According to one embodiment, the (meth)acrylate monomer is at least one of methyl methacrylate, ethyl methacrylate, ethyleneglycol dimethacrylate and butane-1,4-diol dimethacrylate. According to a preferred embodiment, the (meth)acrylate monomer is a C₁-C₁₀-alkyl methacrylate, in one embodiment a C₁-C₆-alkyl methacrylate, and in one embodiment is methyl methacrylate (MMA).

The (meth)acrylate monomer may be present in the electrically conductive adhesive in specific amounts. For example, the (meth)acrylate monomer may be present in the adhesive in an amount in the range of 60 to 90 wt. %, in one embodiment of 65 to 80 wt. %, in one embodiment of 65 to 75 wt. %, and in one embodiment of 68 to 74 wt. %, based on the total weight of the organic compounds in the adhesive. The “total weight of the organic compounds in the adhesive” is to be understood in that the basis of the indicated weight is the sum of the weight of all organic compounds in the adhesive. Organic compounds in this context are initiators, polymers, monomers, organic crosslinkers, organic primers etc. The biocompatible metal is not an organic compound.

The (meth)acrylate monomer may be present in the adhesive in an amount in the range of 5 to 40 wt. %, in one embodiment of 10 to 30 wt. %, in one embodiment of 12 to 20 wt. %, based on the total weight of the adhesive. The (meth)acrylate monomer may be present in the adhesive in an amount in the range of 30 to 85% by volume, in one embodiment of 35 to 75% by volume, in one embodiment of 40 to 75% by volume, based on the total volume of the adhesive.

The electrically conductive adhesive may also include further polymerizable monomers, and in one embodiment radically polymerizable monomers. For example, the electrically conductive monomer may include at least one further monomer being different from the monomer described herein as monomer a), wherein the monomer is selected from the group of (meth)acrylate monomers, (meth)acrylic acid, (meth)acrylamide (e.g. methacrylamide), and mixtures thereof. In one embodiment, the electrically conductive adhesive further includes a (meth)acrylamide and/or a di(meth)acrylate, and in one embodiment further includes methacrylamide and ethyleneglycol dimethacrylate.

The electrically conductive adhesive according to one embodiment includes a polymer being soluble in the (meth)acrylate monomer.

It has been found by the inventors that including the polymer as described herein (herein also referred to as the polymer b)) into the electrically conductive adhesive has several advantages. The polymer may be included into the adhesive to adjust the viscosity and/or internal strength of the adhesive to a suitable value. Furthermore, it has been found that by including the polymer into the adhesive the curing time of the adhesive may be influenced and/or adjusted to a suitable curing time.

The polymer is preferably soluble in the (meth)acrylate monomer in an amount of at least 25 g/L (e.g. in the range of 25 to 600 g/L) at a temperature of 25° C., preferably of at least 50 g/L, and in one embodiment of at least 100 g/L (e.g. in the range of 100 to 600 g/L).

The polymer is in one embodiment a polymer having a weight median molecular weight of below 750 000 g/mol (e.g. of 250 to 750 000 g/mol), in one embodiment of below 500 000 g/mol, in one embodiment of below 200 000 g/mol. The weight median molecular weight of the polymer is in one embodiment determined by a viscometric method.

The polymer is in one embodiment a non-crosslinked polymer.

In one embodiment, the polymer is a biocompatible polymer according to ISO 10993.

The polymer may be a homopolymer or a copolymer. In one embodiment, the polymer is selected from the group consisting of selected from the group consisting of poly((meth)acrylates), poly((meth)acrylate) copolymers, and mixture thereof. In one embodiment, the polymer is selected from the group consisting of selected from the group consisting of poly(methacrylates), polymethacrylate copolymers, and mixture thereof. In one embodiment, the polymer is selected from the group consisting of selected from the group consisting of poly(alkyl methacrylates), poly(alkyl methacrylate) copolymers, and mixture thereof. In one embodiment, the polymer is selected from the group consisting of selected from the group consisting of poly(methyl methacrylates), poly(methyl methacrylate) copolymers, and mixture thereof.

According to one preferred embodiment, the polymer is selected from the group consisting of poly(methyl methacrylates) (PMMA), poly(ethyl methacrylates) (PEMA), poly(propyl methacrylates), poly(isopropyl methacrylates), poly(methyl methacrylate-co-methacrylate), poly(methyl methacrylate-co-styrene), and mixtures thereof. According to a more preferred embodiment, the polymer is a poly(methyl methacrylate) (PMMA). A suitable poly(methyl methacrylate) is Degacryl® MW 332 from Evonik.

The polymer may be present in the adhesive in specific amounts. For example, the polymer may be present in the adhesive in an amount in the range of 10 to 30 wt. %, in one embodiment 12 to 25 wt. %, in one embodiment 15 to 22 wt. %, based on the total weight of the organic compounds in the adhesive.

The polymer may be present in the adhesive in an amount in the range of 0.75 to 12 wt. %, in one embodiment 1.5 to 8 wt. %, in one embodiment 2 to 6 wt. %, based on the total weight of the adhesive. The polymer may be present in the adhesive in an amount in the range of 10 to 30% by volume, in one embodiment of 10 to 25% by volume, based on the total volume of the adhesive.

The electrically conductive adhesive according to one embodiment includes a biocompatible metal having a particle size d₅₀ of below 50 μm. According to a preferred embodiment, the particle size distribution of the metal as indicated herein is a volume-based particle size distribution, which is in one embodiment measured by laser diffraction.

In one embodiment, the biocompatible metal is a biocompatible metal according to ISO 10993.

According to another embodiment, the biocompatible metal is a biocompatible metal according to ISO 10993, which is not gold.

The biocompatible metal is in one embodiment selected from the group consisting platinum, gold, iridium, steel, titanium, hafnium, niobium, tantalum, cobalt, chromium, zirconium, rhenium, tungsten, molybdenum, and alloys of each one of these metals. In one embodiment, the biocompatible metal is selected from the group consisting of platinum, iridium, steel, titanium, hafnium, niobium, tantalum, cobalt, chromium, zirconium, rhenium, tungsten, molybdenum, and alloys of each one of these metals.

According to a preferred embodiment, the biocompatible metal is platinum or a platinum alloy (e.g. a platinum/iridium alloy). In one embodiment, the biocompatible metal is platinum.

The biocompatible metal in one embodiment has a particle size d₅₀ in the range of from 0.01 to 50 μm, in one embodiment from 0.01 to 25 μm, in one embodiment from 0.2 to 10 μm, and in one embodiment from 0.5 to 3 μm. Furthermore, the biocompatible metal in one embodiment has a particle size d₉₀ in the range of from 0.1 to 80 μm, in one embodiment from 0.5 to 40 μm, in one embodiment from 1.0 to 20 μm, and in one embodiment from 1.0 to 8 μm. According to one embodiment, the biocompatible metal has a particle size d₅₀ in the range of from 0.2 to 10 μm, in one embodiment from 0.5 to 3 μm, and a particle size d₉₀ in the range of from 1.0 to 20 μm, in one embodiment from 1.0 to 8 μm.

The biocompatible metal may have a specific surface area in the range of 0.01 to 50 m²/g, in one embodiment 0.4 to 30 m²/g, in one embodiment of 0.5 to 10 m²/g, in one embodiment from 0.5 to 5 m²/g, as measured with nitrogen and the BET method.

The biocompatible metal may include particles having a specific particle geometry such as spheric, needles or plates. In one embodiment, the biocompatible metal includes particles having a needle or flake geometry.

The biocompatible metal is included into the adhesive to render the adhesive electrically conductive. Therefore, it is understood by a skilled person that the biocompatible metal is present in the adhesive in an amount to render the adhesive conductive. The amount of the biocompatible metal which needs to be added to the adhesive to render the adhesive conductive may also be expressed by the percolation threshold. So, the biocompatible metal may be present in the adhesive in an amount above its percolation threshold.

The biocompatible metal may be present in the adhesive in an amount in the range of 50 to 95 wt. %, in one embodiment 60 to 90 wt. %, in one embodiment 70 to 85 wt. %, and in one embodiment in an amount of 73 to 78 wt. % (e.g. 76 to 78 wt. %), based on the total weight of the adhesive.

Furthermore, the biocompatible metal may be present in the adhesive in an amount of from 7.5 to 50% by volume, in one embodiment of from 10 to 50% by volume, in one embodiment of from 12.5 to 50% by volume, in one embodiment from 14 to 30% by volume (e.g. from 14 to 20% by volume), based on the total volume of the adhesive.

According to another embodiment, the biocompatible metal is present in the adhesive in an amount of from 20 to 50% by volume, in one embodiment of from 30 to 50% by volume, based on the total volume of the adhesive.

The electrically conductive adhesive according to one embodiment includes a polymerization initiator.

The polymerization initiator is included into the adhesive to initiate polymerization of the (meth)acrylate monomer as defined herein. The polymerization initiator is in one embodiment stable at a temperature of 25° C.

The polymerization initiator is in one embodiment a radical polymerization initiator. For example, the initiator may be a peroxide, a diazo compound, a barbiturate, or a photoinitiator. Suitable peroxides are e.g. dibenzoyl peroxide or cumolhydroxy peroxide. Suitable diazo compounds are e.g. azobis(isobutyronitrile) (AIBN) or dimethyl-azobis(2-methylpropionate) (V-601). Suitable barbiturates are e.g. selected from the group of 1,5-disubstituted barbiturates, 1,3,5-trisubstituted barbiturates and 1,3,5-tetrasubstituted barbiturates (e.g. selected from the group of 1-cyclohexyl-5-ethyl barbituric acid, 1-phenyl-5-ethyl barbituric acid, 1-benzyl-5-ethyl barbituric acid and 1,3,5-trimethyl barbituric acid). It is preferred that the initiator is a peroxide, in one embodiment dibenzoyl peroxide.

The polymerization initiator may be activated by adding a polymerization activator to the adhesive, by applying heat to the adhesive, and/or by applying UV light to the adhesive. In one embodiment, the initiator can be activated by adding a polymerization activator to the adhesive and/or by applying heat to the adhesive. According to one embodiment, the initiator can be activated by applying heat to the adhesive.

The polymerization initiator may be a compound having a self accelerating decomposition temperature (SADT) in the range of 35 to 100° C., in one embodiment in the range of 50 to 85° C.

The polymerization initiator may be present in the adhesive in an amount in the range of from 0.1 to 3.0 wt. %, in one embodiment from 0.5 to 2.5 wt. %, in one embodiment from 1.2 to 2.4 wt. %, based on the total weight of the organic compounds in the adhesive. Moreover, the polymerization initiator may be present in the adhesive in an amount in the range of from 0.05 to 2.0 wt. %, in one embodiment from 0.1 to 1.5 wt. %, in one embodiment from 0.2 to 1.0 wt. %, based on the total weight of the adhesive. The polymerization initiator may be present in the adhesive in an amount in the range of from 0.02 to 3.0% by volume, in one embodiment from 0.2 to 2.5% by volume, in one embodiment from 1.2 to 2.4 wt. %, based on the total volume of the adhesive.

The components may also be present in the adhesive in specific relative amounts to each other. It is preferred that the adhesive includes the following components in the following relative amounts (with pbw being “parts by weight”):

| | |
|---|---|
| (meth)acrylate | 60 to 100 pbw, in one embodiment 70 to 90 pbw, |
| monomer | |
| polymer b) | 10 to 30 pbw, in one embodiment 16 to 24 pbw, |
| initiator | 1.0 to 3.0 pbw, in one embodiment 1.6 to 2.4 pbw. |

In addition to the components above, the electrically conductive adhesive may include further components. For example, the electrically conductive adhesive may include further polymers (e.g. polymers which are not soluble in the (meth)acrylate monomer), polymerization activators, antibiotics, stabilizers, primers, crosslinkers, tackifying agents, and/or fillers. “A polymer being not soluble in the (meth)acrylate monomer” means that the polymer dissolves with less than 25 g/L, in one embodiment less than 5 g/L, in the monomer at a temperature of 25° C.

According to one embodiment, the electrically conductive adhesive includes a primer and/or a crosslinker. According to one preferred embodiment, the adhesive includes a primer and a crosslinker.

The electrically conductive adhesive in one embodiment includes a crosslinker. The crosslinker may be any kind of crosslinker which is suitable as a crosslinker in poly((meth)acrylate) adhesives (e.g. difunctional or trifunctional compounds). For example, the crosslinker may be a diol di(meth)acrylate (e.g. ethyleneglycol dimethacrylate, butane-1,4-diol dimethacrylate, or hexane-1,4-diol dimethacrylate). In one embodiment, the crosslinker is ethyleneglycol dimethacrylate.

In one embodiment, the crosslinker is present in the adhesive in an amount in the range of 1.0 to 15 wt. %, in one embodiment of 3.0 to 10 wt. %, in one embodiment of 4 to 8 wt. %, based on the total weight of the organic compounds in the adhesive. According to another preferred embodiment, the adhesive includes a crosslinker in an amount in the range of 0.1 to 6 wt. %, in one embodiment of 0.4 to 3 wt. %, based on the total weight of the adhesive. According to another embodiment, the crosslinker is present in the adhesive in an amount in the range of 0.5 to 14% by volume, in one embodiment of 1.5 to 10% by volume, based on the total volume of the adhesive.

The electrically conductive adhesive in one embodiment includes a primer. The primer may be any primer which is suitable for use as a primer in poly((meth)acrylate) adhesives. For example, the primer may be (meth)acrylamide. In one embodiment, the primer is methacrylamide.

In one embodiment, the primer is present in the adhesive in an amount in the range of 0.5 to 5.0 wt. %, in one embodiment of 1.5 to 3.5 wt. %, in one embodiment, of 2.0 to 3.0 wt. %, based on the total weight of the organic compounds in the adhesive. According to another preferred embodiment, the adhesive includes a primer in an amount in the range of 0.05 to 2.5 wt. %, in one embodiment of 0.1 to 1.2 wt. %, based on the total weight of the adhesive. According to another embodiment, the primer is present in the adhesive in an amount in the range of 0.25 to 14% by volume, in one embodiment of 1.0 to 10% by volume, based on the total volume of the adhesive.

According to another preferred embodiment, the adhesive includes the following components in the following relative amounts:

| | |
|---|---|
| (meth)acrylate | 60 to 100 pbw, in one embodiment 70 to 90 pbw, |
| monomer | |
| polymer b) | 10 to 30 pbw, in one embodiment 16 to 24 pbw, |
| initiator | 1.0 to 3.0 pbw, in one embodiment 1.6 to 2.4 pbw, |
| crosslinker | 3.0 to 9.0 pbw, in one embodiment 5.0 to 7.0 pbw, |
| primer | 0.8 to 4.0 pbw, in one embodiment 2.0 to 3.5 pbw. |

The electrically conductive adhesive may include a polymerization activator. The polymerization activator may be an aromatic amine (e.g. N,N-dimethyl-p-toluidine, N,N-bis-hydroxyethyl-p-toluidine, N,N-dimethyl-aniline, 4-N,N-dimethylamino-pyridine), saccharine, lithium chloride, trioctylmethylammonium chlorid, or mixtures thereof. In one embodiment, the activator is an aromatic amine, and particularly N,N-dimethyl-p-toluidine. The foregoing activators are particularly preferred, if the polymerization initiator is a peroxide.

The polymerization activator may also be a copper(II) salt, in one embodiment selected from the group of copper(II) ethyl hexanoate, copper(II) methacrylate, copper(II) acetyl acetonate, basic copper(II) carbonate, copper(II) hydroxide, and mixtures thereof. In one embodiment, the polyimerization activator is a copper(II) salt, if the polymerization initiator is a barbiturate.

The electrically conductive may include a polymerization activator in an amount of 0.05 to 3.0 wt. %, in one embodiment from 0.2 to 2.0 wt. %, based on the total weight of the organic compounds in the adhesive. According to one embodiment, the polymerization activator is present in the adhesive in an amount in the range of from 0.01 to 1.0 wt. %, in one embodiment from 0.02 to 0.5 wt. %, in one embodiment from 0.04 to 0.2 wt. %, based on the total weight of the adhesive. The polymerization initiator may be present in the adhesive in an amount in the range of from 0.01 to 1.5% by volume, in one embodiment from 0.1 to 1.2% by volume, in one embodiment from 0.6 to 1.0 wt. %, based on the total volume of the adhesive.

The polymerization activator is in one embodiment used in a specific relative amount with respect to the polymerization initiator. The polymerization activator is in one embodiment used in a weight ratio to the polymerization initiator in the range of 1:10 to 2:1 [activator:initiator], in one embodiment in the range of 1:6 to 1.5:1, in one embodiment in the range of 1:4 to 1:1.

The electrically conductive adhesive may have an electrical resistance of below 20 Ohm, in one embodiment in the range of 0.05 to 10 Ohm, in one embodiment in the range of 0.1 to 5 Ohm, in one embodiment in the range of 0.1 to 2 Ohm, and in one embodiment in the range of 0.1 to 1 Ohm. The electrical resistance is measured for the cured adhesive e.g. by using a Mulimeter Agilent 34401A.

In one embodiment, the electrically conductive adhesive is an isotropic conductive adhesive.

According to one very preferred embodiment, the electrically conductive adhesive includes

a) a C₁-C₁₀-alkyl methacrylate, in one embodiment methyl methacrylate (MMA),
wherein the C₁-C₁₀-alkyl methacrylate is present in the adhesive in an amount of in an amount in the range of 5 to 40 wt. %, in one embodiment of 10 to 30 wt. %, based on the total weight of the adhesive,

b) a polymer being soluble in the (meth)acrylate monomer, which is selected from the group consisting of poly((meth)acrylates), poly((meth)acrylate) copolymers, and mixture thereof, in one embodiment is poly(methyl methacrylate) (PMMA),
wherein the polymer is present in the adhesive in amount in the range of 0.75 to 12 wt. %, in one embodiment 1.5 to 8 wt. %, based on the total weight of the adhesive,

c) a biocompatible metal having a median particle size d₅₀ of below 50 μm, which is selected from the group consisting of platinum, gold, iridium, steel, titanium, hafnium, niobium, tantalum, cobalt, chromium, zirconium, rhenium, tungsten, molybdenum, and alloys of each one of these metals, in one embodiment is platinum,
wherein the biocompatible metal is present in the adhesive in an amount in the range of 50 to 85 wt. %, in one embodiment 70 to 82 wt. %, and in one embodiment 75 to 80 wt. %, based on the total weight of the adhesive,

d) a polymerization initiator, which is selected from the group consisting of peroxides, barbiturates, and mixture thereof, in one embodiment a peroxide,
wherein the polymerization initiator is present in the adhesive in an amount in the range of from 0.02 to 1.5 wt. %, in one embodiment from 0.1 to 1.0 wt. %, based on the total weight of the adhesive,

e) a crosslinker, in one embodiment selected from the group consisting of diol di(meth)acrylates,
wherein the crosslinker is present in the adhesive in an amount in the range of 0.1 to 6 wt. %, in one embodiment of 0.4 to 3 wt. %, based on the total weight of the adhesive,

f) a primer, in one embodiment methacrylamide,
wherein the primer is present in the adhesive in an amount in the range of 0.05 to 2.5 wt. %, in one embodiment of 0.1 to 1.2 wt. %, based on the total weight of the adhesive.

The electrically conductive adhesive may have a curing time of below 30 min, in one embodiment in the range of 1 to 20 min, in one embodiment in the range of 2 to 15 min.

According to a preferred embodiment, the electrically conductive adhesive is suitable for being used to connect electronic parts in an implantable medical device. In one embodiment, the implantable medical device is an active implantable medical device (AIMD). Preferred AIMDs are, for example, cardiac pacemakers, cardiac defibrillators, neurostimulators, cochlea implants, implantable cardioverters, nerve, brain, organ or muscle stimulators as well as implantable monitoring devices, hearing aids, retinal implants, muscle stimulators, implantable drug pumps, artificial hearts, bone growth stimulators, prostate implants and stomach implants.

### Kit for an Electrically Conductive Adhesive

Another aspect of one embodiment relates to a kit for preparing an electrically conductive adhesive. The kit includes a component A and a component B,

wherein component A includes a (meth)acrylate monomer, and

wherein component B includes a polymer being soluble in the (meth)acrylate monomer of component A, a biocompatible metal having a median particle size d₅₀ of below 50 μm, and a polymerization initiator.

The inventive kit can be used to prepare an electrically conductive adhesive according one embodiment by mixing component A with component B. According to a preferred embodiment, the kit is a kit for preparing an electrically conductive adhesive as defined in the foregoing section.

The foregoing section discloses embodiments, and preferred embodiments, for the (meth)acrylate monomer of the inventive adhesive. The same embodiments, and preferred embodiments, are also disclosed herein in connection with and/or also apply to the (meth)acrylate monomer of component A of the inventive kit. This means that an embodiment of the (meth)acrylate monomer, which is described in the foregoing section, is also an embodiment of the (meth)acrylate monomer of component A of the inventive kit.

The (meth)acrylate monomer is present in component A of the kit. Optionally, the (meth)acrylate monomer is also present in component B of the kit. For example, the (meth)acrylate monomer may be present in component B of the kit, if it is desired that component B is not a solid such as a powder but a paste.

The (meth)acrylate monomer may be present in the kit in specific amounts. For example, the (meth)acrylate monomer may be present in (e.g. in component A) in an amount in the range of 60 to 90 wt. %, in one embodiment of 65 to 80 wt. %, in one embodiment of 65 to 75 wt. %, and in one embodiment of 68 to 74 wt. %, based on the total weight of the organic compounds of components A and B. The (meth)acrylate monomer may be present in the kit (e.g. in component A) in an amount in the range of 5 to 40 wt. %, in one embodiment of 10 to 30 wt. %, in one embodiment of 12 to 20 wt. %, based on the total weight of all components of the kit. The (meth)acrylate monomer may be present in (e.g. in component A) in an amount in the range of 30 to 85% by volume, in one embodiment of 35 to 75% by volume, in one embodiment of 40 to 75% by volume, based on the total volume of all components of the kit.

The foregoing section discloses embodiments, and preferred embodiments, for the polymer, the biocompatible metal powder, and the polymerization initiator of the inventive adhesive. The same embodiments, and preferred embodiments, are also disclosed herein in connection with and/or also apply to the polymer, the biocompatible metal, and the polymerization initiator of component B of the inventive kit. This means that an embodiment of the polymer, the biocompatible metal, or the polymerization initiator, which is described in the foregoing section, is also an embodiment of the polymer, the biocompatible metal, or the polymerization initiator of component B of the inventive kit.

The polymer is present in component B of the kit. Optionally, the polymer is also present in component A of the kit. For example, the polymer may be present in component A of the kit to adjust the viscosity of component A e.g. to a paste-like viscosity.

The polymer may be present in the kit in specific amounts. For example, the polymer may be present in the kit (e.g. in component B) in an amount in the range of 10 to 30 wt. %, in one embodiment 12 to 25 wt. %, in one embodiment 15 to 22 wt. %, based on the total weight of the organic compounds of components A and B of the kit. The polymer may be present in the kit (e.g. in component B) in an amount in the range of 0.75 to 12 wt. %, in one embodiment 1.5 to 8 wt. %, in one embodiment 2 to 6 wt. %, based on the total weight of components A and B of the kit. The polymer may be present in the kit (e.g. in component B) in an amount in the range of 10 to 30% by volume, in one embodiment of 10 to 25% by volume, based on the total volume of components A and B of the kit.

The biocompatible metal may be present in the kit (e.g. in component B) in an amount in the range of 50 to 95 wt. %, in one embodiment 60 to 90 wt. %, in one embodiment 70 to 85 wt. %, and in one embodiment in an amount of 73 to 78 wt. % (e.g. 76 to 78 wt. %), based on the total weight of components A and B of the kit. Furthermore, the biocompatible metal may be present in the kit (e.g. in component B) in an amount of from 7.5 to 50% by volume, in one embodiment of from 10 to 50% by volume, in one embodiment of from 12.5 to 50% by volume, in one embodiment from 14 to 30% by volume (e.g. from 14 to 20% by volume), based on the total volume of components A and B of the kit. According to another embodiment, the biocompatible metal is present in the kit (e.g. component B) in an amount of from 20 to 50% by volume, in one embodiment of from 30 to 50% by volume, based on the total volume of components A and B of the kit.

The polymerization initiator may be present in the kit (e.g. in component B) in an amount in the range of from 0.1 to 3.0 wt. %, in one embodiment from 0.5 to 2.5 wt. %, in one embodiment from 1.2 to 2.4 wt. %, based on the total weight of the organic compounds of components A and B of the kit. Moreover, the polymerization initiator may be present in the kit (e.g. in component B) in an amount in the range of from 0.05 to 2.0 wt. %, in one embodiment from 0.1 to 1.5 wt. %, in one embodiment from 0.2 to 1.0 wt. %, based on the total weight of components A and B of the kit. The polymerization initiator may be present in the kit (e.g. in component B) in an amount in the range of from 0.02 to 3.0% by volume, in one embodiment from 0.2 to 2.5% by volume, in one embodiment from 1.2 to 2.4 wt. %, based on the total volume of components A and B of the kit.

Components A and/or B of the kit may further include further polymers, polymerization activators, antibiotics, stabilizers, primers, crosslinkers, tackifying agents, and/or fillers. Preferred polymerization activators, primers and crosslinkers are described in the foregoing section with respect to the inventive electrically conductive adhesive.

According to a preferred embodiment, component A of the kit includes a polymerization activator. Preferred polymerization activator are described in the foregoing section with respect to the inventive electrically conductive adhesive.

The kit (e.g. component A) may include a polymerization activator in an amount of 0.05 to 3.0 wt. %, in one embodiment from 0.2 to 2.0 wt. %, based on the total weight of the organic compounds in components A and B of the kit. According to one embodiment, the polymerization activator is present in the kit (e.g. component A) in an amount in the range of from 0.01 to 1.0 wt. %, in one embodiment from 0.02 to 0.5 wt. %, in one embodiment from 0.04 to 0.1 wt. %, based on the total weight of components A and B of the kit. The polymerization initiator may be present in the kit (e.g. component A) in an amount in the range of from 0.01 to 1.5% by volume, in one embodiment from 0.1 to 1.2% by volume, in one embodiment from 0.6 to 1.0 wt. %, based on the total volume of components A and B of the kit.

According to a preferred embodiment, component A of the kit further includes a crosslinker and/or a primer, in one embodiment a primer and a crosslinker.

In one embodiment, the crosslinker is present in the kit (e.g. in component A) in an amount in the range of 1.0 to 15 wt. %, in one embodiment of 3.0 to 10 wt. %, in one embodiment of 4 to 8 wt. %, based on the total weight of the organic compounds in components A and B of the kit. According to another preferred embodiment, the kit (e.g. in component A) includes a crosslinker in an amount in the range of 0.1 to 6 wt. %, in one embodiment of 0.4 to 3 wt. %, based on the total weight of components A and B of the kit. According to another embodiment, the crosslinker is present in the kit (e.g. in component A) in an amount in the range of 0.5 to 14% by volume, in one embodiment of 1.5 to 10% by volume, based on the total volume of components A and B.

In one embodiment, the primer is present in the kit (e.g. in component A) in an amount in the range of 0.5 to 5.0 wt. %, in one embodiment of 1.5 to 3.5 wt. %, in one embodiment, of 2.0 to 3.0 wt. %, based on the total weight of the organic compounds in components A and B of the kit. According to another preferred embodiment, the kit (e.g. in component A) includes a primer in an amount in the range of 0.05 to 2.5 wt. %, in one embodiment of 0.1 to 1.2 wt. %, based on the total weight of components A and B of the kit. According to another embodiment, the primer is present in the kit (e.g. in component A) in an amount in the range of 0.25 to 14% by volume, in one embodiment of 1.0 to 10% by volume, based on the total volume of components A and B of the kit.

According to a preferred embodiment, the kit is a kit for preparing an electrically conductive adhesive, which is suitable for being used to connect electronic parts in an implantable medical device. In one embodiment, the implantable medical device is an active implantable medical device (AIMD). Preferred AIMDs are, for example, cardiac pacemakers, cardiac defibrillators, neurostimulators, cochlea implants, implantable cardioverters, nerve, brain, organ or muscle stimulators as well as implantable monitoring devices, hearing aids, retinal implants, muscle stimulators, implantable drug pumps, artificial hearts, bone growth stimulators, prostate implants and stomach implants.

### Implantable Medical Device

In another aspect of one embodiment, an implantable medical device is provided. The implantable medical device includes at least two electronic parts,

wherein the at least two electronic parts are connected by

a) the electrically conductive adhesive according to the present embodiments, or a cured form thereof, or

b) the electrically conductive adhesive prepared from the kit according to the present embodiments, or a cured form thereof.

A skilled person understands that a cured form of the electrically conductive adhesive of the present embodiments includes a polymer which is derived from the (meth)acrylate monomer of the adhesive. The cured form of the adhesive further includes the biocompatible metal, which renders the cured form of the adhesive electrically conductive. The cured form of the adhesive further includes the polymer of the inventive adhesive, or a reaction product thereof. If at all, the cured form of the adhesive only includes the polymerization initiator in a significantly lower amount than the non-cured form of the adhesive. According to a preferred embodiment, the at least two electronic parts are connected by a cured form of the electrically conductive adhesive.

According to one preferred embodiment, the at least two electronic parts are connected by the electrically conductive adhesive according to the present embodiments, or a cured form thereof. According to another embodiment, the at least two electronic parts are connected by the electrically conductive adhesive prepared from the kit according to the present embodiments, or a cured form thereof According to a preferred embodiment, the at least two electronic parts are connected by a cured form of the electrically conductive adhesive.

According to a preferred embodiment, the implantable medical device is an active implantable medical device (AIMD). Preferred AIMDs are, for example, cardiac pacemakers, cardiac defibrillators, neurostimulators, cochlea implants, implantable cardioverters, nerve, brain, organ or muscle stimulators as well as implantable monitoring devices, hearing aids, retinal implants, muscle stimulators, implantable drug pumps, artificial hearts, bone growth stimulators, prostate implants and stomach implants.

The at least two electronic parts are not particularly limited. The at least two electronic parts may be independently selected from the group of wires, electrical feedthroughs (e.g. cermet feedthroughs), electrodes, microelectrodes, electrode arrays, flexible electrode arrays, electronic leads, electronic terminals, and contact elements.

According to one preferred embodiment, the at least two electronic parts are a wire and an electrode array (e.g. a flexible electrode array). According to another preferred embodiment, the at least two electronic parts are a wire and an electrical feedthrough (e.g. cermet feedthrough). A “cermet feedthrough” is a feedthrough including a ceramic component and a metal component. Such feedthroughs are known to the skilled person. According to yet another preferred embodiment, the at least two electronic parts are an electrode (e.g. a microelectrode) and a lead.

### Use and/or Process

Another aspect of one embodiment relates to a use of an electrically conductive adhesive, or use of a kit according to one embodiment, for connecting at least two electronic parts in an implantable medical device.

Thus, one embodiment refers to the use of a kit according for connecting at least two electronic parts in an implantable medical device. One preferred embodiment refers to a use of an electrically conductive adhesive according to one embodiment for connecting at least two electronic parts in an implantable medical device.

According to a preferred embodiment, the implantable medical device is an active implantable medical device (AIMD). Preferred AIMDs are, for example, cardiac pacemakers, cardiac defibrillators, neurostimulators, cochlea implants, implantable cardioverters, nerve, brain, organ or muscle stimulators as well as implantable monitoring devices, hearing aids, retinal implants, muscle stimulators, implantable drug pumps, artificial hearts, bone growth stimulators, prostate implants and stomach implants.

The at least two electronic parts are not particularly limited. The at least two electronic parts may be independently selected from the group of wires, electrical feedthroughs (e.g. cermet feedthroughs), electrodes, microelectrodes, electrode arrays, flexible electrode arrays, electronic leads, electronic terminals, and contact elements.

According to one preferred embodiment, the at least two electronic parts are a wire and an electrode array (e.g. a flexible electrode array). According to another preferred embodiment, the at least two electronic parts are a wire and an electrical feedthrough (e.g. cermet feedthrough). A “cermet feedthrough” is a feedthrough including a ceramic component and a metal component. Such feedthroughs are known to the skilled person. According to yet another preferred embodiment, the at least two electronic parts are an electrode (e.g. a microelectrode) and a lead.

Another aspect of one embodiment relates to a process for connecting at least two electronic parts in an implantable medical device by using of the electrically conductive adhesive according to one embodiment, or by using the kit according to one embodiment.

According to one embodiment, the process uses the electrically conductive adhesive according to one embodiment. In such case, the process may include the steps of 1) applying the electrically conductive adhesive according to one embodiment to a first electronic part, 2) contacting the applied electrically conductive adhesive to the second electronic part, and 3) curing the electrically conductive adhesive.

According to a preferred embodiment, the process uses the kit according to one embodiment. In such case, the process may include the steps of 1) mixing component A with component B of the kit to prepare an electrically conductive adhesive, 2) applying the electrically conductive adhesive to a first electronic part, 3) contacting the applied electrically conductive adhesive to the second electronic part, and 4) curing the electrically conductive adhesive.

The features disclosed in the claims and the specification may be essential for different embodiments of the claimed embodiments, both separately and in any combination with each other. In the following, the claimed embodiments are further described by an example. The example is not understood to be limiting the claimed embodiments in any way.

### EXAMPLE

TABLE 1
Materials

| | |
|---|---|
| (Meth)acrylate monomer | Methyl methacrylate (CAS: 80-62-6) |
| Polymer (soluble in the | Polymethyl methacrylate (PMMA), |
| monomer) | Degacryl ® MW 332, commercially available |
| | from Evonik |
| Biocompatible metal | Platinum powder, Pt M579B, commercially |
| | available from Heraeus, surface area 0.8-2.0 |
| | m²/g; tap density 3.0-4.0; particle size: d₉₀ = |
| | 1.5-3.0; d₅₀ = 0.9-1.9, d₁₀ = 0.5-0.9. |
| Polymerization initiator | Dibenzoyl peroxide (CAS: 94-36-0) |
| Polymerization activator | Dimethyl-p-toluidine (CAS: 99-97-8) |
| Crosslinker | Ethyleneglycol dimethacrylate (CAS: 97-90-5) |
| Primer | Methacrylamide (CAS: 79-39-0) |
| Substrate | Silver-coated polyimide |

A PMMA solution was prepared by mixing the components as indicated in Table 2 with each other, and stirring the mixture over night.

TABLE 2
PMMA solution

| Methyl | | Dibenzoyl | | Ethyleneglycol |
|---|---|---|---|---|
| methacrylate | PMMA | peroxide | Methacrylamide | dimethacrylate |
| (wt. %) | (wt. %) | (wt. %) | (wt. %) | (wt. %) |
| 72.8 | 18 | 1.8 | 2.4 | 6 |

Table 3 shows the recipe of a conductive adhesive according to one embodiment. The inventive adhesive was prepared by mixing the metal powder into the PMMA solution to obtain a paste, followed by adding the polymerization activator to the mixture. The paste was handled and/or applied manually by using a spatula.

TABLE 3
Inventive adhesive

| Metal powder (wt. %) | PMMA solution (wt. %) | Activator (wt. %) |
|---|---|---|
| 77.98 | 21.98 | 0.04 |

The inventive adhesive was analyzed for its curing time, electrical resistance and its lap shear strength on a silver-coated polyimide substrate. The results are shown in Table 4.

TABLE 4
Properties of inventive adhesive

| | | | Electrical |
|---|---|---|---|
| | Lap shear strength | Electrical | resistance |
| Curing time | according to DIN | resistance | Ø (10 |
| (min) | EN 53 283 (N/mm²) | (Ω) | measurements) |
| 7 | 5.63 (failure of silver | 0.5 to 1.5 | 1 |
| | coating on polyimide) | | |

The conductive adhesive showed a lap shear strength according to DIN EN 53 283 of at least 5.63 N/mm². At this shear strength, the silver coating on the polyimide substrate failed so that the actual shear strength of the adhesive is presumably higher than the measured value.

The electrical resistance is an indicator for the electrical conductivity of the adhesive. The average electrical resistance of the adhesive was 1 Ω, which indicates a good electrical conductivity.

Although specific embodiments have been illustrated and described herein, it will be appreciated by those of ordinary skill in the art that a variety of alternate and/or equivalent implementations may be substituted for the specific embodiments illustrated and described without departing from the scope of the present embodiment. This application is intended to cover any adaptations or variations of the specific embodiments discussed herein. Therefore, it is intended that this embodiment be limited only by the claims and the equivalents thereof.

1. An electrically conductive adhesive comprising:
a) a (meth)acrylate monomer,
b) a polymer being soluble in the (meth)acrylate monomer,
c) a biocompatible metal having a median particle size d₅₀ of below 50 μm, and
d) a polymerization initiator.

2. The electrically conductive adhesive according to claim 1, wherein the (meth)acrylate monomer is a C₁-C₁₀-alkyl methacrylate, and is methyl methacrylate (MMA).

3. The electrically conductive adhesive according to claim 1, wherein the polymer is selected from the group consisting of poly((meth)acrylates), poly((meth)acrylate) copolymers, poly(methyl methacrylates) (PMMA), poly(ethyl methacrylates) (PEMA), poly(propyl methacrylates), poly(isopropyl methacrylates), poly(methyl methacrylate-co-methacrylate), poly(methyl methacrylate-co-styrene), and mixtures thereof.

4. The electrically conductive adhesive according to claim 1, wherein the biocompatible metal is selected from the group consisting of platinum, gold, iridium, steel, titanium, hafnium, niobium, tantalum, cobalt, chromium, zirconium, rhenium, tungsten, molybdenum, and alloys of each one of these metals.

5. The electrically conductive adhesive according to claim 1, wherein the biocompatible metal has a particle size d₅₀ in the range of from 0.5 to 3 μm, or wherein the biocompatible metal has a particle size d₉₀ in the range of from 1.0 to 8 μm.

6. The electrically conductive adhesive according to claim 1, wherein the biocompatible metal is present in the adhesive in an amount in the range of 50 to 95 wt. %, based on the total weight of the adhesive, or wherein the biocompatible metal is present in the adhesive in an amount of from 7.5 to 50% by volume, based on the total volume of the adhesive.

7. The electrically conductive adhesive according to claim 1, wherein the biocompatible metal is present in the adhesive in an amount in the range of 70 to 85 wt. %, based on the total weight of the adhesive, or wherein the biocompatible metal is present in the adhesive in an amount of from 12.5 to 50% by volume, based on the total volume of the adhesive.

8. The electrically conductive adhesive according to claim 1, wherein the (meth)acrylate monomer is present in the adhesive in an amount in the range of 65 to 75 wt. %, based on the total weight of the organic compounds in the adhesive, or
wherein the polymer is present in the adhesive in an amount in the range of 12 to 25 wt. %, based on the total weight of the organic compounds in the adhesive, or
wherein the polymerization initiator is present in the adhesive in an amount in the range of from 0.5 to 2.5 wt. %, based on the total weight of the organic compounds in the adhesive.

9. The electrically conductive adhesive according to claim 1, wherein the adhesive comprises a primer in an amount in the range of from 1.5 to 3.5 wt. %, based on the total weight of the organic compounds in the adhesive.

10. The electrically conductive adhesive according to claim 1, wherein the adhesive comprises a crosslinker in an amount in the range of from 3.0 to 10 wt. %, based on the total weight of the organic compounds in the adhesive.

11. The electrically conductive adhesive according to claim 1, wherein the adhesive comprises a polymerization activator in an amount of from 0.2 to 2.0 wt. %, based on the total weight of the organic compounds in the adhesive.

12. The electrically conductive adhesive according to claim 1, wherein the polymerization initiator is a compound having a self-accelerating decomposition temperature (SADT) in the range of from 50 to 85° C., or wherein the polymerization initiator is a peroxide, a diazo compound, a barbiturate, or a photoinitiator.

13. The electrically conductive adhesive according to claim 1, wherein the adhesive comprises the following components in the following relative amounts:

| | |
|---|---|
| (meth)acrylate monomer | 60 to 100 pbw, |
| polymer b) | 10 to 30 pbw, |
| initiator | 1.0 to 3.0 pbw, |
| optionally a crosslinker | 3.0 to 9.0 pbw, |
| optionally a primer | 0.8 to 4.0 pbw. |

14. The electrically conductive adhesive according to claim 1, wherein the adhesive comprises the following components in the following relative amounts:

| | |
|---|---|
| (meth)acrylate monomer | 70 to 90 pbw, |
| polymer b) | 16 to 24 pbw, |
| initiator | 1.6 to 2.4 pbw, |
| optionally a crosslinker | 5.0 to 7.0 pbw, |
| optionally a primer | 2.0 to 3.5 pbw. |

15. A kit for preparing an electrically conductive adhesive,
wherein the electrically conductive adhesive comprises a (meth)acrylate monomer, a polymer being soluble in the (meth)acrylate monomer, a biocompatible metal having a median particle size d₅₀ of below 50 μm, and a polymerization initiator,
wherein the kit comprises a component A and a component B,
wherein component A comprises the (meth)acrylate monomer, and
wherein component B comprises the polymer being soluble in the (meth)acrylate monomer of component A, the biocompatible metal having a median particle size d₅₀ of below 50 μm, and the polymerization initiator.

16. An implantable medical device comprising at least two electronic parts,
wherein the at least two electronic parts are connected by the electrically conductive adhesive according to claim 1, or a cured form thereof.

17. An implantable medical device comprising at least two electronic parts,
wherein the at least two electronic parts are connected by the electrically conductive adhesive prepared from the kit according to claim 15, or a cured form thereof.

18. Use of an electrically conductive adhesive according to claim 1, for connecting at least two electronic parts in an implantable medical device.

19. Use of a kit according to claim 15, for connecting at least two electronic parts in an implantable medical device.
